(19) United States
(12) Patent Application Publication
(10) Pub. No.: US 2025/0218618 A1
(43) Pub. Date: Jul. 3, 2025

(54) ENERGY MODULATING DEVICE, PARTICLE BEAM IRRADIATING DEVICE USING THE SAME, PARTICLE BEAM IRRADIATING METHOD, AND PARTICLE-BEAM RADIATION THERAPY PLANNING DEVICE

(71) Applicant: NATIONAL INSTITUTES FOR QUANTUM SCIENCE AND TECHNOLOGY, Chiba-shi, Chiba (JP)

(72) Inventors: Sodai TANAKA, Chiba-shi, Chiba (JP); Taku INANIWA, Chiba-shi, Chiba (JP)

(21) Appl. No.: 18/686,648

(22) PCT Filed: Jul. 29, 2022
(86) PCT No.: PCT/JP2022/029372

§ 371 Date: Mar. 5, 2025

(30) Foreign Application Priority Data
Aug. 31, 2021 (JP) 2021-141156

(51) Int. Cl. G21K 1/10; A61N 5/10; G21K 1/093

(52) U.S. Cl. CPC G21K 1/10; A61N 5/1043; A61N 5/1071; G21K 1/093; A61N 2005/1095

(57) ABSTRACT

Provided are a particle beam irradiating device capable of reducing a distance between an irradiation field forming device of the particle beam irradiating device and a patient, as well as an energy modulating device used for the particle beam irradiating device. The energy modulating device, i.e. a ripple filter 7, used for the particle beam irradiating device 10 that transports, by a beam transport line 2, a charged particle beam extracted from an accelerator 1 and delivers the charged particle beam by a scanning method using scanning magnets 4 and 5. The energy modulating device, i.e. the ripple filter 7, includes a filter member 70 having a plurality of openings 72 which penetrate the filter member 70 in a thickness direction and through which at least a part of the charged particle beam passes, in which the filter member 70 is one of two or more filter members overlapped in the thickness direction.

### TECHNICAL FIELD

The present invention relates to a particle beam irradiating device for treating cancer or the like using a proton beam or charged particle radiation, a particle beam irradiating method, a particle-beam radiation therapy planning device, and an energy modulating device used for these.

### BACKGROUND ART

Conventionally, in cancer treatment, a particle beam irradiating device that takes out charged particle radiation (charged particle beam) mainly including a proton beam and a carbon ion beam from an accelerator and irradiates a tumor (subject to be irradiated) with the charged particle beam has been used.

As such a particle beam irradiating device, there has been proposed a broad beam type particle beam irradiating system that enlarges an irradiation field of a charged particle beam to be delivered in X and Y directions, increases an energy width (Bragg peak) of the charged particle beam in a Z direction, and causes a collimator to narrow the irradiation field to a predetermined shape for irradiation (see Patent Literature 1).

Regarding this particle beam irradiating system, an energy distribution forming device for forming a predetermined energy distribution has been proposed. The energy distribution forming device is configured such that a rod-shaped body as a first energy absorber and a columnar-shaped body as a second energy absorber are arranged on a virtual plane of a ridge filter. The rod-shaped body has a stepped portion whose thickness changes stepwise in the X direction. With this energy distribution forming device, it is considered that an enlarged Bragg curve having a large spread-out Bragg peak (SOBP) width with high accuracy can be produced, the production can be facilitated, and to the production cost can be suppressed. Further, such a particle beam irradiating device delivers a charged particle beam having an energy distribution adjusted by a ridge filter after adjusting the charged particle beam to the shape of the tumor by the collimator.

As shown in FIG. 11 of Patent Literature 1 as the conventional example, a ridge filter (ripple filter) having only a rod-shaped body is generally used in many cases. In addition, there is a scanning type particle beam irradiating system that, unlike the broad beam type, scans a thin pencil beam by an electromagnet to fill a cancer target with a Bragg peak, but the ridge filter used can be common.

However, when the ridge filter as described above is used, unevenness occurs in scattering of the charged particle beam after passing through the ridge filter, and a distance between an irradiation target and the ridge filter increases when a position of the irradiation target is matched with a point where the unevenness of scattering is reduced. That is, there is a problem that a distance required between the patient and the irradiation field forming device (irradiation unit) of the particle beam irradiating device becomes long.

### CITATION LIST

### Patent Literature

Patent Literature 1: JP 2010-117257 A

### SUMMARY OF INVENTION

### Technical Problem

In view of the above-described problems, an object of the present invention is to provide an energy modulating device capable of reducing a distance between an irradiation unit of a particle beam irradiating device and a patient, as well as a particle beam irradiating device, a particle beam irradiating method, and a particle-beam radiation therapy planning device using the energy modulating device.

### Solution to Problem

The present invention provides an energy modulating device used for a particle beam irradiating device that transports, by a beam transport line, a charged particle beam extracted from an accelerator and delivers the charged particle beam by a scanning method using a scanning magnet, the energy modulating device including a filter member having a plurality of openings which penetrate the filter member in a thickness direction and through which at least a part of the charged particle beam passes, in which the filter member is one of two or more filter members overlapped in the thickness direction.

### Advantageous Effects of Invention

According to the present invention, a distance between an irradiation field forming device of a particle beam irradiating device and a patient can be shortened.

### BRIEF DESCRIPTION OF DRAWINGS

FIG. 1 is a configuration diagram of a particle beam irradiating device and a particle-beam radiation therapy planning device.

FIG. 2 is a configuration diagram of an irradiation unit.

FIG. 3 is an explanatory diagram for illustration of a configuration of a filter member.

FIG. 4 is an explanatory diagram for illustration of the way in which filter members are overlapped.

FIG. 5 is an explanatory diagram in which filter members are actually overlapped.

FIG. 6 is a configuration diagram in which three filter members are overlapped.

FIGS. 7A and 7B are explanatory diagrams for illustration of a range of an opening portion of FIGS. 7A and 7B.

FIG. 8 is a distribution diagram showing distribution in a depth direction (irradiation direction) of a relative dose in a case where a ripple filter of this example is used.

FIG. 9 is a distribution diagram showing distribution in the depth direction (irradiation direction) of a relative dose due to a change in the number of filter members.

FIG. 10 is a distribution diagram showing distribution in the depth direction (irradiation direction) of a relative dose due to a change in a position of the ripple filter.

FIG. 11 is a dose distribution diagram measured by a dosimetry film.

### DESCRIPTION OF EMBODIMENT

Hereinafter, an embodiment of the present invention will be described with reference to the drawings.

### Examples

### <Particle Beam Irradiating Device>

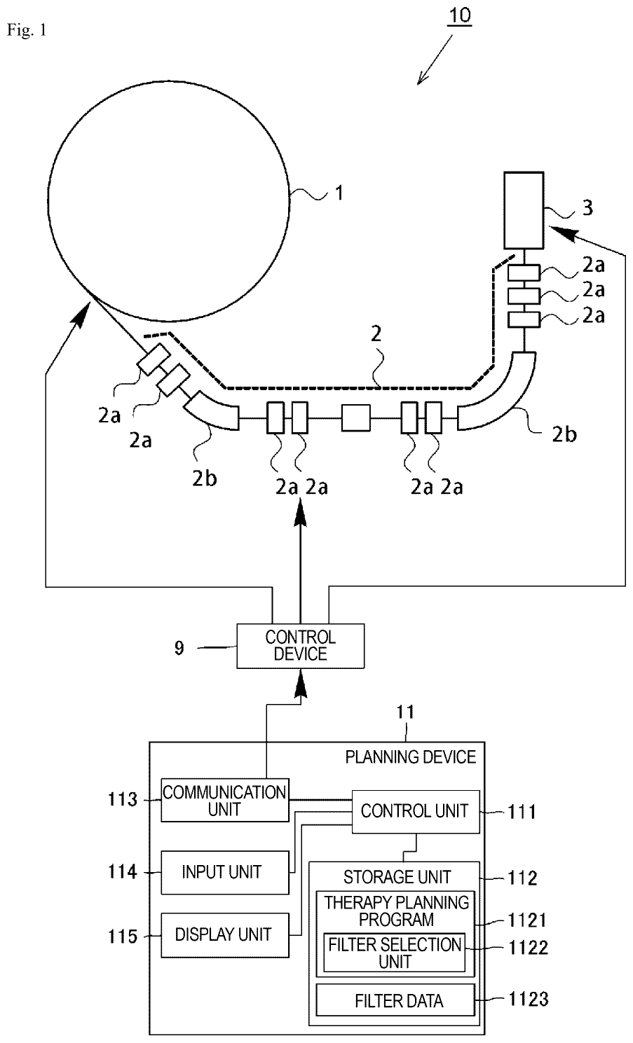

FIG. 1 is a configuration diagram illustrating a schematic configuration of a particle beam irradiating device 10 and a particle-beam radiation therapy planning device.

The particle beam irradiating device 10 mainly includes an accelerator 1, a beam transport line 2, and an irradiation unit 3 (irradiation field forming device and beam irradiation unit), and the particle-beam radiation therapy planning device includes a control device 9 that controls the particle beam irradiating device 10 and a planning device 11 that transmits a particle-beam radiation therapy plan to the control device 9.

The accelerator 1 accelerates a charged particle beam extracted from an ion source using various electromagnets, a high-frequency acceleration device, or the like. The beam transport line 2 includes a vacuum duct, a quadrupole electromagnet 2a, a deflecting electromagnet 2b, and the like, and transports the charged particle beam extracted from the accelerator 1.

The irradiation unit 3 is provided at an end of the beam transport line 2.

As a result, the charged particle beam extracted from the ion source is accelerated by the accelerator 1 and then transported to the irradiation unit 3 via the beam transport line 2.

### <Irradiation Unit>

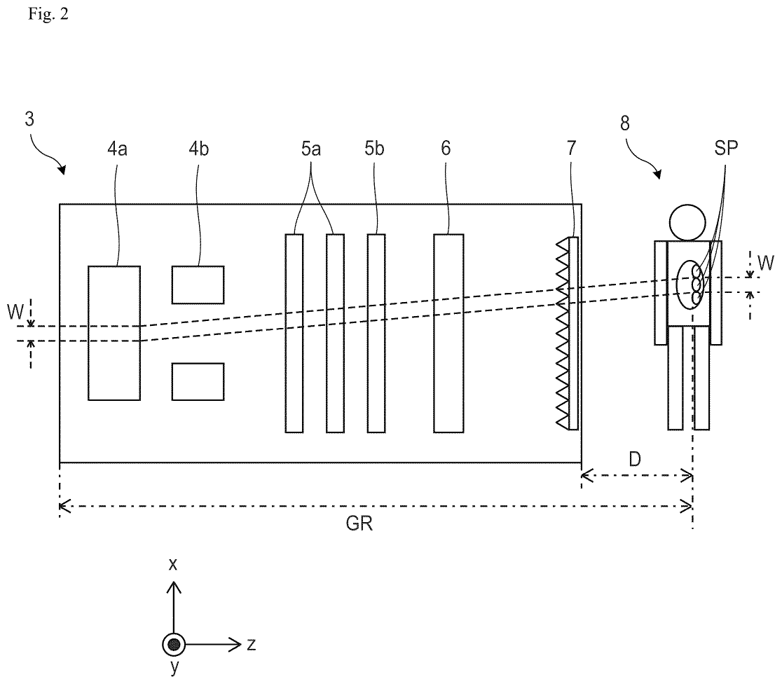

FIG. 2 is a configuration diagram of the irradiation unit 3.

The charged particle beam transported to the irradiation unit 3 has a particle number distribution according to a Gaussian distribution, and a beam size W is not particularly limited, but can be set to general setting in which σ in the Gaussian distribution (normal distribution) is about 2-3 mm.

Then, when an irradiation dose applied to a certain irradiation point measured by a dose monitor 5a reaches a set value for the irradiation point, the irradiation unit 3 controls an X-direction scanning magnet 4a and a Y-direction scanning magnet 4b, changes a stop position of the charged particle beam in a Z direction (a traveling direction of the charged particle beam) by an energy changing unit 6, or changes an output of the emitted beam energy of the accelerator 1, and changes and outputs the dose distribution of the charged particle beam by a ripple filter 7. As a result, the irradiation position of the charged particle beam can be scanned to the next irradiation point. The irradiation position of the charged particle beam scanned at the next irradiation point is monitored by the beam position monitor 5b.

In this manner, the irradiation unit 3 three-dimensionally controls and scans (performs scanning) the charged particle beam, and sequentially irradiates each of irradiation spots SP three-dimensionally arranged according to the shape of the tumor in the body of the patient 8 as a subject to be irradiated with the charged particle beam.

### <Energy Modulating Device (Ripple Filter)>

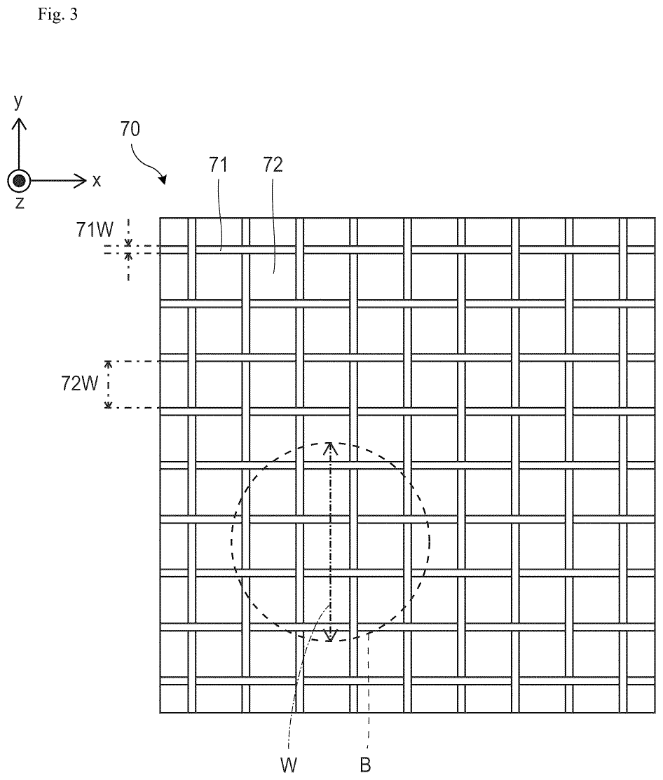

FIG. 3 is an explanatory view for illustration of a configuration of a filter member 70 constituting the ripple filter 7. The ripple filter 7 includes a plurality of filter members 70. Each of the filter members 70 has a thin plate shape as a whole, and includes a filter material 71 through which at least a part of a charged particle beam B passes and an opening 72 through which at least a part of the charged particle beam B passes. In this example, as the filter member 70, a net-shaped filter member as an example of a thin plate shape was used, and specifically, a 10-cm square stainless mesh plate made of the filter material 71 of stainless steel (SUS 304) was used.

The filter material 71 is made of a material that changes the dose distribution of the charged particle beam B after transmission as viewed from a transmission direction of the charged particle beam B transmits (the transmission direction of the charged particle beam B or the traveling direction of the charged particle beam B). The amount of change in the dose distribution of the charged particle beam B is evaluated by a stopping power ratio (an effective thickness of a substance relative to the charged particle beam). The stopping power ratio of the filter material 71 can be set to 0.8 or more when water is 1.0, and is preferably set to 2.0 or more. The filter material 71 is preferably made of metal, as having a sufficient stopping power ratio, more preferably made of iron, steel, or aluminum among metals, and still more preferably made of aluminum. In this example, stainless steel (SUS 304) having a stopping power ratio of 5.4 was used as the filter material 71.

The filter material 71 in this example is a wire rod having a uniform wire diameter (thickness), and a plurality of filter materials are provided on an XY plane (plane perpendicular to the traveling direction of the charged particle beam) of the filter member 70. Further, the plurality of filter materials 71 have the same wire diameter, and are linearly disposed at regular intervals in the X direction and the Y direction and are parallel to the X direction and the Y direction. A maximum wire diameter 71W (width of the filter material) of the filter material 71 is preferably 0.5 mm or less, and more preferably 0.2 mm or less. The filter material 71 includes a filter material extending in the Y direction (vertically) and a filter material extending in the X direction (horizontally), and has a plain weave structure in which a plurality of filter materials 71 extending in the Y direction and a plurality of filter materials 71 extending in the X direction alternately intersect. In other words, the stainless mesh plate is formed in a net shape in which a plurality of cylindrical wire rods having a regularly waving shape (filter materials 71) are arranged in parallel in a longitudinal direction and a lateral direction and combined. An amplitude and a period of the wave of the cylindrical wire rods are all the same, and intervals of the plurality of wire rods arranged in parallel are regularly arranged at equal intervals in both the vertical and horizontal directions. Intervals between the plurality of filter materials 71 extending in the Y direction may be at least larger than the maximum wire diameter 71W, and may be equal intervals or may be random. Further, intervals between the plurality of filter materials 71 extending in the X direction may be at least larger than the maximum wire diameter of 71W, and may be equal intervals or may be random. Furthermore, the intervals between the plurality of filter materials 71 extending in the Y direction and the intervals between the plurality of filter materials 71 extending in the X direction may all be equal intervals (the same distance). Further, the wire diameters of the plurality of filter materials 71 disposed in one filter member 70 may not be the same, and the wire diameters of one filter material 71 may not be uniform.

The opening 72 is defined in a space surrounded by the filter material 71 extending in the Y direction and the filter material 71 extending in the X direction. That is, the opening 72 is defined by the filter materials 71. In the present embodiment, the opening 72 has a substantially square shape when viewed from the Z direction. A maximum value of an opening side 72W indicating the length of the side of the opening 72a is smaller than the beam size W of the charged particle beam B. The minimum value of the opening side 72W is preferably larger than the maximum wire diameter 71W. It is preferable that a plurality of openings 72 having the same shape are regularly provided on the XY plane of the filter member 70. Further, the ratio (opening ratio) of the area of the opening 72 to the area of the filter member 70 on the XY plane can be set to 50% or more, preferably 60% or more, more preferably 70% or more, and still more preferably 80% or more.

The filter member 70 in this example is a stainless mesh plate formed in a lattice shape by the filter material 71 made of stainless steel (SUS 304) regularly arranged in the X direction and the Y direction and the square openings 72 formed so as to be surrounded by the filter material 71 and regularly arranged. In this example, a stainless mesh plate having a maximum wire diameter of 71W of 0.1 mm and one side (interval between adjacent filter materials 71) of the opening 72a of 0.508 mm was used.

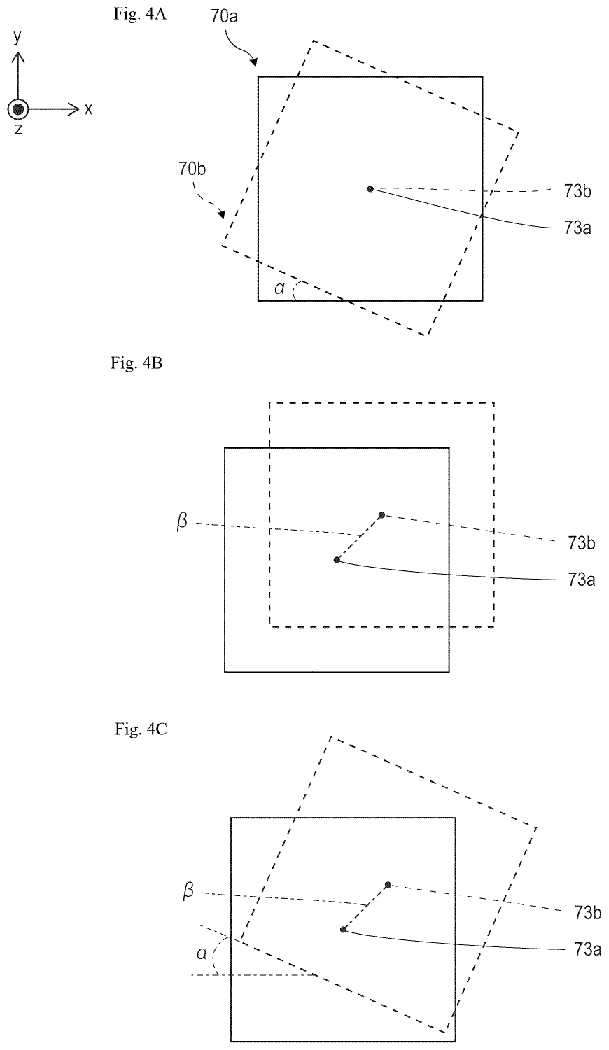

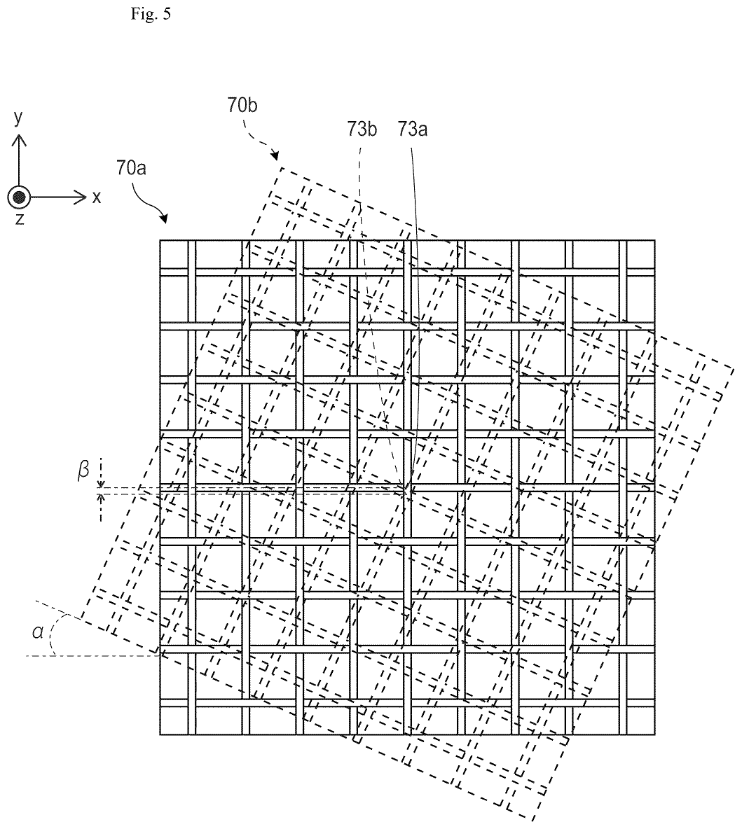

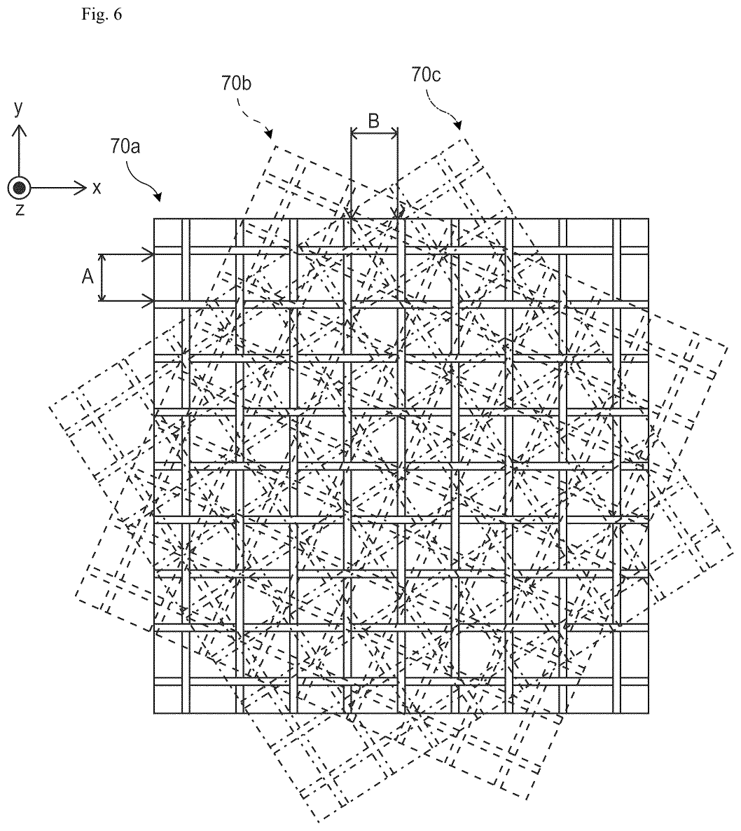

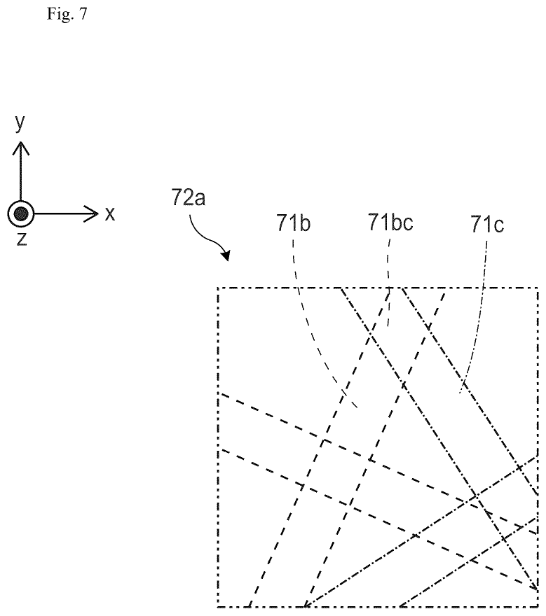

FIG. 4 is an explanatory diagram for illustration of the way in which two filter members 70a and 70b are overlapped, FIG. 5 is an explanatory diagram in which the two filter members 70a and 70b are actually overlapped, FIG. 6 is a configuration diagram in which three filter members 70a, 70b, and 70c are overlapped, and FIG. 7 is an explanatory diagram for illustration of a range of the opening portion of A-B shown in FIG. 6.

The ripple filter 7 is configured such that a plurality of filter members 70 are arranged in an overlapping manner in the traveling direction of the charged particle beam. The plurality of filter members 70 are configured such that, within a range of the opening 72 of one filter member 70 as viewed in the traveling direction of the charged particle beam (the Z direction, or a thickness direction of the ripple filter 7), at least one filter material 71 of other filter members 70 is disposed.

Each of the plurality of filter members 70 is overlapped with another filter member 70 with at least one of a position and an angle in a planar direction being shifted from one another. For example, as shown in FIG. 4a, a filter material 71a parallel to the X direction of the first filter member 70a and a filter material 71b parallel to the X direction of the second filter member 70b are rotated by an angle α (center points 73a and 73b overlap). Alternatively, as illustrated in FIG. 4b, the center point 73a of the first filter member 70a is moved by a distance β in at least one of the X direction and the Y direction with respect to the center point 73b of the second filter member 70b. Alternatively, as illustrated in FIG. 4c, both the rotation of the angle α and the movement of the distance β are performed. In each of the filter members 70a, 70b in FIGS. 4a, 4b, and 4c, the filter material 71 and the opening 72 are not shown so that the center points 73a and 73b can be easily seen. However, the distance β is different from an integral multiple of the length of opening side 72W of the opening 72, and the rotation angle α is larger than 0 degrees and smaller than 90 degrees. In addition, it is preferable that both the state of being rotated by the angle α and the state of being moved by the distance β are satisfied. In this example, as illustrated in FIG. 5, the first filter member 70a and the second filter member 70b are arranged so as to satisfy both the rotation of the angle α and the movement of the distance β.

In this example, the filter member 70 is configured such that the filter materials 71 are arranged at equal intervals and the openings 72 are regularly arranged. However, for example, when the intervals between the filter materials 71 are random in the X direction or the Y direction, the filter materials may not be moved with respect to the X direction or the Y direction in which the filter materials are randomly arranged, or may not be rotated on the XY plane.

The third filter member 70c does not exactly overlap with both the first filter member 70a and the second filter member 70b. That is, the position of the third filter member 70c is shifted by β2 in the X direction or the Y direction with respect to the second filter member 70b, or rotated by an angle α2 formed by the filter material 71b of the second filter member 70b parallel to the X direction and the filter material 71c of the third filter member 70c parallel to the X direction. However, it is preferable that after the third filter member 70c is moved or rotated, all the filter materials 71a of the first filter member 70a and all the filter materials 71c of the third filter member 70c do not exactly overlap each other.

Hereinafter, similarly, the fourth and subsequent filter members 70 are shifted in distance in the X direction or the Y direction, or rotated on the XY plane, or both of them are satisfied and overlapped. The number of overlapping filter members 70 can be 3 or more, and is preferably 5 or more, and more preferably 10 or more. The filter material 71 of two thirds or more out of the plurality of overlapping filter members 70 does not exactly overlap with the filter material 71 of another filter member 70. Further, the ratio at which filter materials 71 do not exactly overlap with each other is preferably 70% or more, and more preferably 80% or more. In addition, it is preferable that two or more filter members 70 constituting the ripple filter 7 have a planar shape (flat plate shape) and are arranged in parallel to each other. In this example, the filter member 70 has a planar shape (flat plate shape), and two or more filter members 70 overlapped in the thickness direction are overlapped with flat surfaces being in contact with each other. That is, although the two or more filter members 70 are arranged in parallel to each other, the present invention is not limited to such an example, and the two or more filter members 70 may not be in contact with each other or may not be arranged in parallel.

In the ripple filter 7 in which the plurality of filter members 70 are overlapped in this manner, when the charged particle beam B is transmitted, the number of overlaps of the wire rods of the ripple filter 7 and the distance of transmission through the wire rods are different depending on the positions. Therefore, the distance of movement in the filter member 70 for each charged particle present in the charged particle beam B is random, and the high dose region (Bragg peak) after transmission through the ripple filter 7 increases. More specifically, the plurality of charged particles included in the charged particle beam B passes through the filter material 71 of the filter member 70 constituting the ripple filter 7. At this time, every time the charged particles pass through one filter material 71, the distance to the high dose region becomes shorter. The number (thickness) of the filter material 71 that transmits the charged particles varies, and the number of overlaps of the wire rods of the ripple filter 7 and the distance that transmits the wire rods vary depending on the positions. Therefore, the moving distance of each charged particle in the filter member 70 becomes random. Therefore, after the transmission through the ripple filter 7, the timing at which the charged particles have a high dose randomly changes for each charged particle depending on the moving distance in the filter member 70, and the dose distribution of the charged particles included in the charged particle beam B becomes random. That is, when viewed from the traveling direction of the charged particle beam, the thickness of the ripple filter 7 is randomly formed, and the high dose region (Bragg peak) of the charged particle beam B after transmission increases. In particular, in this example, since the filter material 71 is a wire rod having a circular cross section, there are a charged particle beam B having a long transmission distance that transmits through the center of the circular shape and a charged particle beam B having a short transmission distance that transmits through the vicinity of the outer periphery of the circular shape, and the change in the energy loss amount varies depending on the position. Since the plurality of ripple filters 7 formed of the filter material 71 are arranged at different positions and/or at different angles, the range of change in the energy loss amount of the charged particle beam B depending on the position in the irradiation range of the delivered charged particle beam B is increased, and the high dose region (Bragg peak) of the transmitted charged particle beam B increases. In this example, the overlapping is performed such that there is almost no position where the filter material 71 of the ripple filter 7 does not exist in a range in which the charged particle beam B can be delivered when viewed from an irradiation direction of the charged particle beam B. When there is a region where the filter material 71 of the ripple filter 7 does not exist in the range in which the charged particle beam B is delivered as viewed from the irradiation direction of the charged particle beam B, a proportion of the region (area) where the filter material does not exist with respect to the range in which the charged particle beam B can be delivered is preferably 1% or less, more preferably 0.5% or less, and still more preferably 0.1% or less.

In this example, the second filter member 70b overlapped with the first filter member 70a is overlapped in a manner such that the movement of least one of the angle α and the distance β is performed. The angle α is formed by rotating the second filter member 70b by a degrees with respect to the first filter member 70a. The distance β is formed by moving the center point 73b of the second filter member 70b by β on the XY plane with respect to the center point 73a of the first filter member 70a. Only one of the angle α and the distance β may be moved, but it is preferable that both the angle α and the distance β are moved. Further, in this example, the distance β is moved only in the Y direction, but may be moved only in the X direction, or in both the X direction and the Y direction.

The third filter member 70c is further overlapped by moving the angle α2 and/or the distance β2. At this time, the third filter member 70c is preferably set such that both the angle α2 and the distance β2 are different from both the angle α and the distance β moved by the second filter member 70b.

As described above, when the plurality of filter members 70 are arranged with their angles and positions shifted from each other, as illustrated in FIG. 7, in a range of one opening 72a of the first filter member 70a, there is an overlapping position 71bc where the filter material 71b of the second filter member 70b, a filter material 71c of the third filter member 70c, and the filter materials 71b and 71c overlap each other. That is, in the range of at least one opening 72 in the filter member 70, there is an overlapping position where the filter materials 71 of the other overlapped filter members 70 overlap for different numbers of sheets (have different numbers of overlaps).

FIGS. 6 and 7 illustrate an example in which three filter members 70 are overlapped, but the number of overlapping filter members 70 is increased to increase the overlapping positions. It is preferable that one or more filter materials 71 exist in the range of one opening 72 at any position. In addition, the number of overlapping filter members 70 can be 3 or more, and is preferably 5 or more, and more preferably 10 or more. This is because the overlapping positions increase as the number of filter members 70 increases.

The ripple filter 7 produced as described above is incorporated inside the irradiation unit 3 as illustrated in FIG. 2, or installed outside the irradiation unit 3 although not illustrated, and is arranged between the energy changing unit 6 and the patient 8 in either case. At this time, in this example, they are arranged perpendicular to the Z direction (parallel to the XY plane), but may be arranged inclined so as to have an angle with respect to the XY plane. However, the thickness direction of the ripple filter 7 must not be perpendicular to the traveling direction of the charged particle beam (must be less than) 90°. That is, the opening 72 intersects with the traveling direction of the charged particle beam (an angle between a virtual plane formed by the opening end of the opening 72 and the traveling direction of the charged particle beam is not) 0°. The XY plane angle of the ripple filter 7 with respect to the traveling direction of the charged particle beam (an angle formed by the traveling direction of the charged particle beam and the ripple filter 7) is preferably 5° or more. In addition, a distance D between the ripple filter 7 and the patient 8 can be set to 0 as long as possible, but the influence of the secondary radiation and the influence of slight unevenness can be reduced by setting the distance D to 5 cm or more. The distance D is more preferably 10 cm or more.

### <Particle-Beam Radiation Therapy Planning Device>

The particle-beam radiation therapy plan as used herein is, when performing the particle therapy on a certain patient, to perform one or more optimizations among performing optimization of dose calculation in the patient body and irradiation parameters, performing optimization of dose calculation in the high dose region, the irradiation target position, and the irradiation parameters, and performing optimization of the ripple filter 7 to be used from the irradiation parameters, in order to deliver the set target dose using filter data of the ripple filter 7 to be used and dose distribution in a unit beam of the charged particle beam determined from the irradiation parameters including emitted energy of the set charged particle beam. Examples of the irradiation parameters here include an intensity of the charged particle beam emitted from the accelerator 1, position correction of the charged particle beam in the beam transport line 2, and control of the beam stop position by the X-direction scanning magnet 4a, the Y-direction scanning magnet 4b, or the energy changing unit 6 of the irradiation unit 3.

Referring back to FIG. 1, the planning device 11 is a computer including a control unit 111, a storage unit 112, a communication unit 113, an input unit 114, and a display unit 115, and functions as an irradiation planning device or a particle-beam radiation therapy planning device. The storage unit 112 stores various programs such as a therapy planning program 1121 and various data such as filter data 1123 to be applied to a therapy plan. The control unit 111 operates using the data of the storage unit 112 according to a program such as the therapy planning program 1121. By this operation, the planning device 11 creates data for the irradiation parameters and the ripple filter 7, and transmits the data for the irradiation parameters and the ripple filter 7 to the control device 9. The input unit 114 includes an input device such as a keyboard and a mouse, and receives an input operation. The display unit 115 includes a display device that displays characters and images such as a display, and displays various images such as a CT image, an MRI image, and a PET image, and modeling images of charged particle beams of various regions (GTV, CTV, PTV) including a high dose region.

The therapy planning program 1121 includes a filter selection unit 1122 that receives selection of the ripple filter 7 to be used. The filter selection unit 1122 receives setting of the ripple filter 7 included in the filter data 1123. The planning device 11 transmits the received filter data 1123 and irradiation parameter data to the control device 9 as a particle-beam radiation therapy plan. The filter data 1123 includes, for example, an identification ID allocated for each filter setting, parameters such as a shape, a material, an aperture ratio, and a wire diameter of the filter member 70 or the filter material 71, and parameters of the ripple filter 7 such as the number of filter materials 71. In place of or in addition to the parameters (parameters such as shape, material, aperture ratio, and wire diameter) of the filter member 70 or the filter material 71, the filter data 1123 may include parameters of the size, position, and dose magnitude of a high dose region (Bragg peak) when the charged particle beam passes through the ripple filter 7.

The control unit 111 models the dose distribution in the unit beam of the charged particle beam from the set filter data 1123 and irradiation parameters of the ripple filter 7. The modeling is performed by using variables registered in advance in the planning device 11 or input. As the variables used for modeling, for example, all variables in the particle beam irradiating device 10 such as parameters such as the shape, the material, the aperture ratio, and the wire diameter of the filter member 70 or the filter material 71 stored in the filter data 1123, parameters of the ripple filter 7 such as the number of the filter materials 71, and irradiation parameters such as the emitted energy of the input charged particle beam are used.

In addition, the control unit 111 executes calculation (optimization calculation) for the dose distribution and displays the modeling result and the calculation result on the display unit 115. The calculation of the dose distribution includes, for example, calculation of the sum of the unit beams determined by the ripple filter 7 and the irradiation parameter. Further, as the optimization calculation for the dose distribution, there are calculation of optimum irradiation parameters for the irradiation position and the irradiation dose of the charged particle beam in a certain irradiation target and the performance of the ripple filter 7, and calculation of optimum performance of the ripple filter 7 in a certain irradiation target and irradiation parameters. That is, the filter selection unit 1122 corresponds to an energy modulating device setting unit, and the control unit 111 corresponds to a modeling unit, a dose calculation unit, and an optimization calculation unit.

When performing the particle-beam radiation therapy, the control device 9 replaces the ripple filter 7 to be used and controls the irradiation parameters of the charged particle beam based on a particle-beam radiation therapy plan received from the planning device 11.

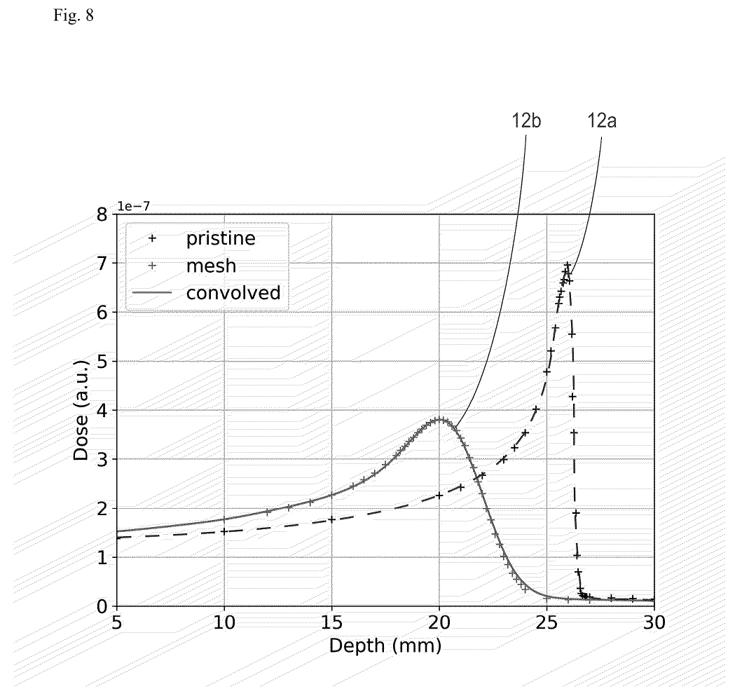

FIG. 8 is a distribution diagram showing distribution in a depth direction (irradiation direction) of a relative dose in a case where the ripple filter 7 of this example is used.

In this example, the ripple filter 7 was produced by overlapping 30 stainless mesh plates with shifted angles and positions.

In a graph illustrated in FIG. 8, the vertical axis represents the dose of the charged particle beam, and the horizontal axis represents the depth (distance) of the charged particle beam in water. This graph shows a post-filter transmission depth dose distribution 12b indicating distribution of dose and depth of the charged particle beam incident on the ripple filter 7 and transmitted through the ripple filter 7, and a raw depth dose distribution 12a indicating distribution of dose and depth of the charged particle beam delivered without transmitting the ripple filter 7. As for the post-filter transmission depth dose distribution 12b, a marker “+” indicates the post-filter transmission depth dose distribution, and a solid line indicates distribution obtained by convolution integral calculation.

As illustrated in FIG. 8, a high dose region (depth region indicated by the horizontal axis with high dose indicated by the vertical axis, Bragg peak) of the charged particle beam that has passed through the ripple filter 7 is wider than a high dose region of the charged particle beam that has not passed through the ripple filter 7. This indicates that an energy loss amount of the charged particle beam transmitted through the ripple filter 7 varies depending on a transmission position of the ripple filter 7. That is, the ripple filter 7 of this example has a role of generating random energy loss for the transmitted charged particle beam and expanding the high dose region.

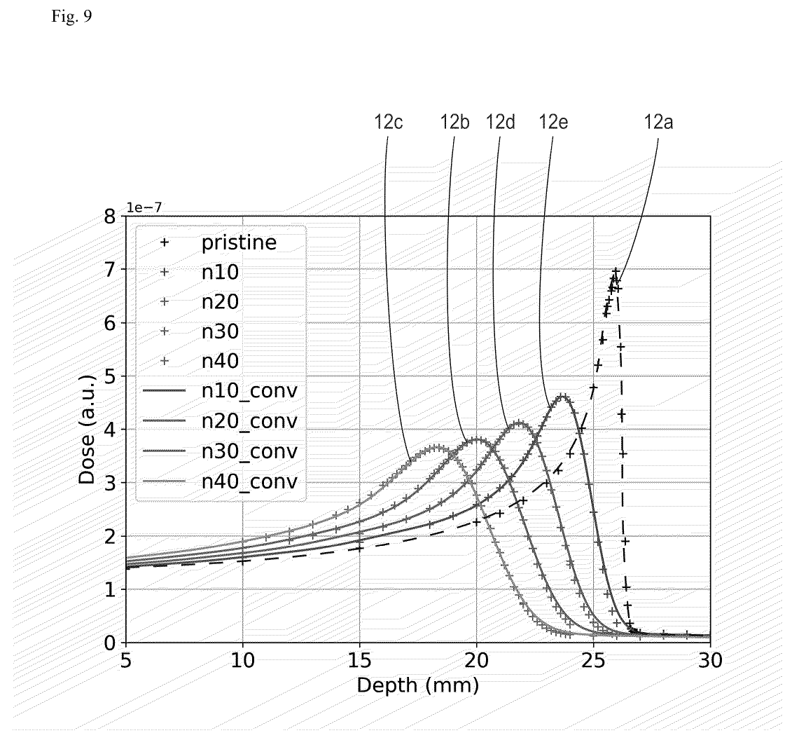

FIG. 9 is a distribution diagram showing distribution in the depth direction (irradiation direction) of a relative dose due to a change in the number of the filter members 70.

In this example, the ripple filter 7 in which the number of filter members 70 is n and n=10, 20, 30, and 40 is produced.

The graph shown in FIG. 9 shows the raw depth dose distribution 12a indicating the distribution of dose and depth of the charged particle beam delivered without passing through the ripple filter 7, a 30 sheet post-filter transmission depth dose distribution 12b with n=30, a 40 sheet post-filter transmission depth dose distribution 12c with n=40, a 20 sheet post-filter transmission depth dose distribution 12d with n=20, and a 10 sheet post-filter transmission depth dose distribution 12e with n=10.

As illustrated in FIG. 9, when the value of n is increased, the high dose region is increased and the distance from the ripple filter 7 is decreased. This indicates that as the number of filter members 70 constituting the ripple filter 7 increases, the amount of energy loss of the transmitted charged particle beam increases, and the randomness of the energy loss due to the difference in the transmission position of the ripple filter 7 increases. That is, the size and the position of the high dose region (Bragg peak) can be changed depending on the number of filter members 70.

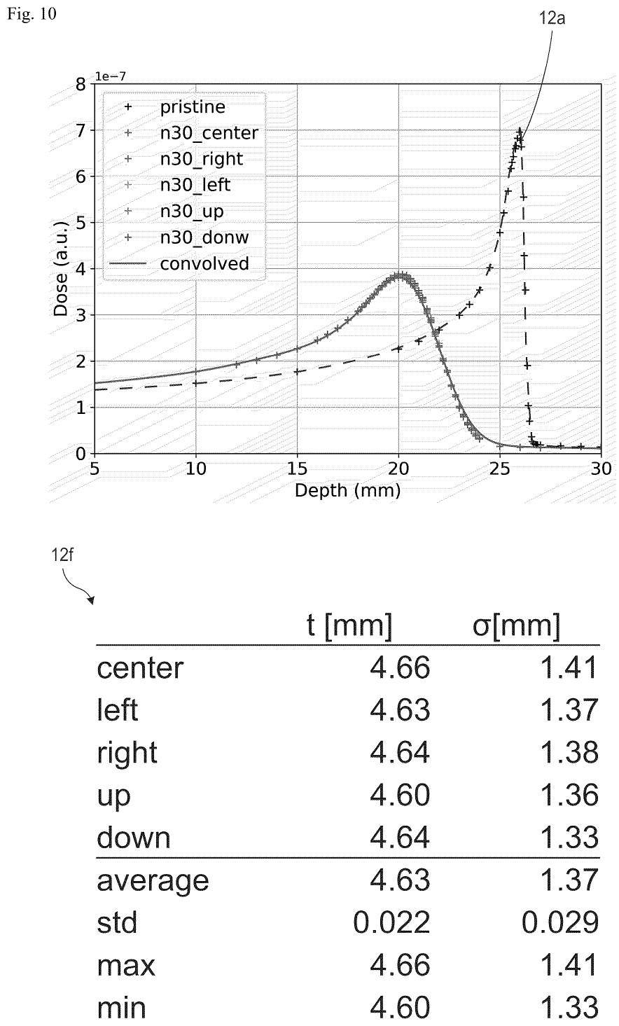

FIG. 10 is a distribution diagram showing distribution in the depth direction (irradiation direction) of a relative dose due to a change in a position of the ripple filter 7 on the XY plane.

In this example, the ripple filter 7 produced by overlapping 30 stainless mesh plates with shifted angles and positions was used.

FIG. 10 illustrates a moving depth dose distribution for each movement pattern in which the ripple filter 7 is moved in an X positive direction, an X negative direction, a Y positive direction, and a Y negative direction on the XY plane and not moved, and the raw depth dose distribution 12a indicating distribution of dose and depth of the charged particle beam delivered without passing through the ripple filter 7.

In addition, a high dose region change table 12f shows σ representing a spreading effect of the high dose region and t representing a change amount (range shift amount) in the depth direction of the high dose region when the ripple filter 7 is moved in the X positive direction, the X reverse direction, the Y positive direction, and the Y reverse direction on the XY plane and not moved. Here, σ indicating a spreading effect of the high dose region and t indicating an amount of change (range shift amount) in the depth direction of the high dose region are defined by [Expression 1] using a planar integrated dose distribution Bm in which the ripple filter 7 is transmitted, a planar integrated dose distribution Bp in which the ripple filter 7 is not transmitted, and the Gaussian function F.

$${B}_{m} ( s ) = {\int }_{- ∞}^{∞} f ( {t}^{′} \vert σ , t ) {B}_{p} ( s + {t}^{′} ) {dt}^{′} \qquad [ Expression 1 ]$$

As shown in each moving depth dose distribution and the high dose region change table 12f, the dose of the charged particle beam after the transmission through the ripple filter 7, the depth of the high dose region, and the size of the range of the high dose region hardly change due to the movement of the ripple filter 7 on the XY plane. That is, regardless of the position of the ripple filter 7 through which the charged particle beam passes, the dose in the high dose region of the same charged particle beam, the depth of the same high dose region, and the size of the range of the same high dose region can be obtained.

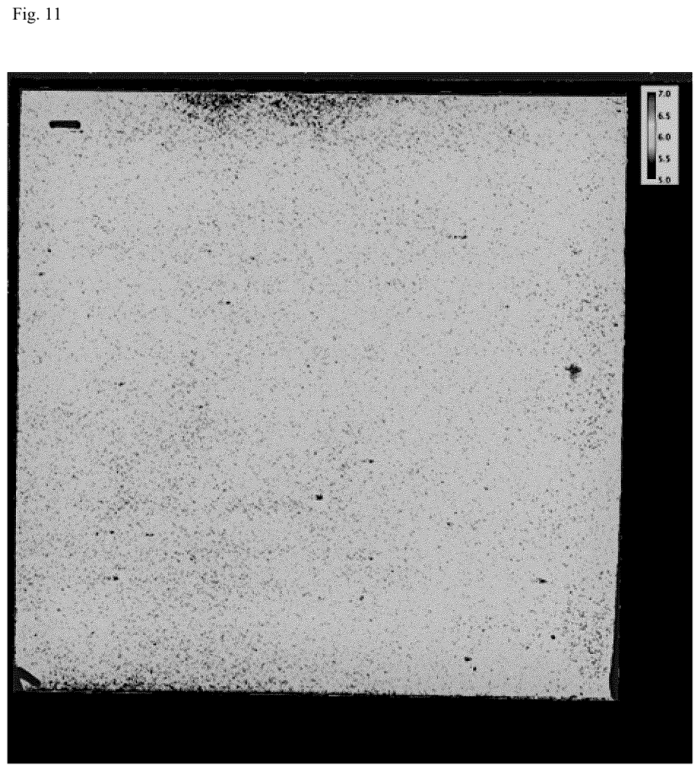

FIG. 11 is a dose distribution diagram in which dose unevenness is measured by a dosimetry film.

In this example, the ripple filter 7 produced by overlapping 30 stainless mesh plates with shifted angles and positions was used.

FIG. 11 is a dose distribution diagram (dose distribution image) obtained by visualizing the dose distribution of the charged particle beam after transmission through the ripple filter 7 with black and white shading, in which a gas chromic film (dosimetry film) of 5 cm on one side is arranged at a distance of 18 cm from the ripple filter 7 on a side of the traveling direction of the charged particle beam.

As shown in FIG. 11, there is almost no black-and-white shading on the gas chromic film. This indicates that there is almost no unevenness on the XY plane in the dose of the charged particle beam after passing through the ripple filter 7.

With the above configuration, it is possible to provide the particle beam irradiating device 10 capable of reducing the distance between the irradiation field forming device (irradiation unit) of the particle beam irradiating device 10 and the patient.

The ripple filter 7 is formed by overlapping two or more filter members 70 having a plurality of openings. With this configuration, the thickness of the ripple filter 7 in one opening becomes random. That is, the energy loss amount of the transmitted charged particle beam is random depending on the transmission position, the high dose region (Bragg peak) in the traveling direction (depth direction) of the transmitted charged particle beam can be increased, and it is possible to suitably use as a ripple filter. Since the randomness of the thickness is fine, the distance D between the ripple filter 7 and the patient 8 (the irradiation spot SP which is the irradiation target present in the body of the patient 8) can be reduced.

The filter member 70 is disposed such that at least one or more filter materials 71 of other filter members 70 overlapped within the range of one opening 72 exist as viewed from the traveling direction of the charged particle beam. With this configuration, the energy loss amount of the transmitted charged particle beam becomes random depending on the transmission position, and the high dose region (Bragg peak) in the traveling direction (depth direction) of the transmitted charged particle beam can be increased, and the filter members 70 can be suitably used as a ripple filter.

Since the ripple filter 7 is formed by overlapping a plurality of filter members 70 having the same shape, the ripple filter 7 can be manufactured at low cost. In particular, since the net-like filter members 70 are overlapped while changing one or both of the angle and the position, the filter members can be easily and inexpensively manufactured without special processing, and a commercially available plain-woven wire mesh can also be used as the filter member 70.

Further, the filter member 70 is configured such that the number of overlaps of the filter material 71 at a certain position in the range of the opening 72 is different from the number of overlaps of the filter material 71 at another overlapping position in the range of the opening 72 when viewed in the traveling direction (axial direction) of the charged particle beam. With this configuration, the distance in which the charged particle beam passes through the ripple filter 7 (the thickness of the ripple filter 7) has fine randomness in a very narrow range. Compared to the conventional ripple filter, the randomness due to the difference in the fine thickness is increased, so that the unevenness can be reduced in the dose distribution in the XY plane of the charged particle beam immediately after the transmission. That is, the distance D between the ripple filter 7 and the patient 8 (the irradiation spot SP as the irradiation target present in the body of the patient 8) can be further reduced. Specifically, as compared with a conventional ripple filter, the distance to the patient can be reduced by 65 cm, and the patient can be brought infinitely close.

Further, a plurality of openings 72 having the same shape are regularly provided on the XY plane of the filter member 70. With this configuration, the thickness of the ripple filter 7 having higher randomness can be provided, and the unevenness of the dose in the XY plane of the transmitted charged particle beam can be further reduced. In addition, since the variation in the thickness of the ripple filter 7 is uniformly provided while having randomness, the dose of the charged particle beam, the depth of the high dose region, and the size of the range of the high dose region can be obtained even if the charged particle beam passes through any position on the XY plane.

Further, each of the plurality of overlapping filter members 70 is overlapped with another filter member 70 with at least one of a position and an angle in the XY direction being shifted from one another. With this configuration, the thickness of the ripple filter 7 having higher randomness can be provided, and the unevenness of the dose in the XY plane of the transmitted charged particle beam can be further reduced. Furthermore, by shifting both the position and the angle in the XY plane direction and overlapping, the thickness of the ripple filter 7 having higher randomness can be formed.

Further, the filter material 71 has a stopping power ratio of 0.8 or more when water is 1.0. With this configuration, the energy of the transmitted charged particle beam can be sufficiently lost, and the depth of the high dose region can vary. Furthermore, by setting the number to 2.0 or more, the energy of the transmitted charged particle beam can be sufficiently lost even when the number of overlapping filter members 70 is small, and the depth of the high dose region can vary.

Further, the filter material 71 has maximum wire diameter 71W of 0.5 mm or less. With this configuration, the thickness of the ripple filter 7 having higher randomness can be provided, and the unevenness of the dose distribution in the XY plane of the transmitted charged particle beam can be further reduced.

Further, in the opening 72, the opening side 72W is provided to be smaller than the beam size W, and the ratio of the area of the filter member 70 to the area on the XY plane (opening ratio) is 50% or more. With this configuration, the thickness of the ripple filter 7 having higher randomness can be provided, and the unevenness of the dose distribution in the XY plane of the transmitted charged particle beam can be further reduced.

Further, the number of overlapping filter members 70 is 3 or more, and more preferably 10 or more. With this configuration, the thickness of the ripple filter 7 having higher randomness can be provided, and the unevenness of the dose distribution in the XY plane of the transmitted charged particle beam can be further reduced.

In addition, by changing one or more of the number of overlapping filter members 70, the wire diameter, the aperture ratio, and the material, the size, the position, and the magnitude of the dose of the high dose region (Bragg peak) can be freely set. That is, even when the beam size W and the energy at the time of emission of the charged particle beam have already been set in a certain particle beam irradiating device, the optimal ripple filter 7 can be easily set for the particle beam irradiating device by appropriately changing one or more of the number of filter members 70, the wire diameter, the aperture ratio, and the material in the ripple filter 7 with respect to the spreading effect of the high dose region and the change amount (range shift amount) in the depth direction of the high dose region determined by the beam size W and the energy at the time of emission of the charged particle beam.

In addition, the ripple filter 7 of the present invention can be used for a particle-beam radiation therapy planning device. A user of the particle beam irradiating device 10 can confirm the effect of the ripple filter 7 by viewing the dose distribution in the unit beam of the charged particle beam modeled by variables such as parameters such as shape, material, aperture ratio, and wire diameter of the filter member 70 or the filter material 71 stored in the filter data 1123 input to the particle-beam radiation therapy planning device, parameters of the ripple filter 7 such as the number of filter materials 71, and irradiation parameters such as the emitted energy of the input charged particle beam. Further, the user of the particle beam irradiating device 10 can select the ripple filter 7 having optimum performance for a certain irradiation target from the irradiation parameters determined in advance.

Note that the present invention is not limited to the above-described example, and can take various forms.

For example, although a stainless steel mesh plate made of stainless steel is used as the filter member 70 in the example, the material of the filter material 71 is not limited to stainless steel, and various materials can be used as long as the energy loss of the transmitted charged particle beam is generated (the stopping power ratio is provided). As such a material, for example, other metals, plastics, and the like can be used. Further, in the plurality of filter members 70, one or more filter members 70 may be made of different materials, or one part of the filter material 71 may be made of different materials in one filter member.

In addition, in the example, the filter member 70 is a stainless mesh plate configured by the filter material 71 of stainless steel (SUS 304) provided in a lattice shape at regular intervals and the square openings 72 provided so as to be regularly arranged in the filter material 71, but may have various forms as long as the filter material 71 and the openings 72 are provided. For example, a punching metal plate having a plurality of circular openings (circular holes) in an aluminum plate may be used as the filter member 70. Further, a plate member in which a plurality of slit-shaped holes is arranged in parallel may be used as the filter member 70. In addition, instead of the circular holes and the slit-shaped holes, the openings may be provided as recesses by etching. Also in these cases, by changing one or both of the position and the angle of the punching metal plate and overlapping a plurality of punching metal plates, the same effect as that of the plain-woven wire mesh can be obtained.

Further, in this example, the control device 9 is configured to control replacement of the ripple filter 7, but the replacement of the ripple filter 7 may be performed by a person involved in the particle-beam radiation therapy by checking the display unit 115.

### INDUSTRIAL APPLICABILITY

The present invention can be used for a particle beam irradiating device that delivers a charged particle beam by a scanning method, and an energy modulating device used in the particle beam irradiating device.

### REFERENCE SIGNS LIST

1 accelerator
2 beam transport line
3 irradiation unit
4a X-direction scanning magnet
4b Y-direction scanning magnet
7 ripple filter
70 filter member
71 filter material
72 opening
71bc overlapping position
10 particle beam irradiating device
W beam size
D width of charged particle beam to be stopped in depth direction

1. An energy modulating device used for a particle beam irradiating device that transports, by a beam transport line, a charged particle beam extracted from an accelerator and delivers the charged particle beam by a scanning method using a scanning magnet, the energy modulating device comprising
a filter member having a plurality of openings through which at least a part of the charged particle beam passes, the openings penetrating the filter member in a thickness direction, wherein
the filter member is one of two or more filter members overlapped in the thickness direction.

2. The energy modulating device according to claim 1, wherein
the filter member includes a filter material, and
at least one filter material of remaining of the two or more filter members is disposed within a range of the openings of the filter member as viewed in a traveling direction of the charged particle beam.

3. The energy modulating device according to claim 2, wherein
the filter member includes an overlapping position in the range of the openings, the overlapping position being a position at which two or more filter materials of the remaining of the filter members overlap in the traveling direction of the charged particle beam, and
as viewed in the traveling direction of the charged particle beam, a number of overlapping filter materials at a certain position within a range of one of the openings is different from a number of overlapping filter materials at another overlapping position that is different from the certain position within the range of the opening.

4. The energy modulating device according to claim 2, wherein the filter member includes the plurality of openings that have the same shape and that are regularly arranged.

5. The energy modulating device according to claim 2, wherein each of the plurality of filter members is overlapped with another filter member with at least one of a position and an angle in a planar direction being shifted from one another.

6. A particle beam irradiating device comprising:
an accelerator that accelerates a charged particle beam;
a beam transport line that transports the charged particle beam extracted from the accelerator; and
a beam irradiation unit that delivers the charged particle beam, wherein
the beam irradiation unit includes the energy modulating device according to claim 1.

7. A particle beam irradiating method comprising:
accelerating charged particles by an accelerator that accelerates a charged particle beam;
transporting the charged particle beam extracted from the accelerator by a beam transport line; and
delivering an irradiation target with the charged particle beam by a beam irradiation unit, wherein
the beam irradiation unit includes the energy modulating device according to claim 1, and
the number of the filter members constituting the energy modulating device is changed according to the irradiation target.

8. A particle-beam radiation therapy planning device comprising:
an energy modulating device setting unit that sets the energy modulating device according to claim 1;
a modeling unit that models a dose distribution of the charged particle beam transmitted through the energy modulating device set by the energy modulating device setting unit;
a dose calculation unit that calculates the dose distribution of the charged particle beam; and
an optimization calculation unit that optimizes a particle-beam radiation therapy plan based on the dose distribution calculated by the dose calculation unit.
